(19) United States
(12) Patent Application Publication
(10) Pub. No.: US 2024/0400971 A1
(43) Pub. Date: Dec. 5, 2024

(54) CELL CULTURE SYSTEM AND CELL CULTURE METHOD

(71) Applicants: CANON KABUSHIKI KAISHA, Tokyo (JP); CANON MEDICAL SYSTEMS CORPORATION, Tochigi (JP)

(72) Inventors: HISAFUMI EBISAWA, Tokyo (JP); YOJI YAMAMOTO, Tokyo (JP); ICHIRO HARADA, Kanagawa (JP)

(21) Appl. No.: 18/665,676

(22) Filed: May 16, 2024

(30) Foreign Application Priority Data
Jun. 2, 2023 (JP) 2023-091759

(51) Int. Cl. C12M 1/00; C12M 1/34

(52) U.S. Cl. CPC C12M 47/02; C12M 23/02; C12M 41/36

(57) ABSTRACT

Provided are a cell culture system and a cell culture method that facilitate estimation of the number of cells after culture. The cell culture system includes: a culture substrate in which a plurality of depressions are arranged and to which cells are caused to adhere; a scraper configured to detach cells adhering to a region other than the depressions on the culture substrate by sliding on the culture substrate and being brought into contact with the region other than the depressions on the culture substrate; and a cell removal mechanism configured to remove, from the culture substrate, the cells detached by the scraper.

### BACKGROUND OF THE INVENTION

### Field of the Invention

The present invention relates to a cell culture system and a cell culture method.

### Description of the Related Art

In general, culturing of adherent cells includes a step of seeding a cell suspension into a flat Petri dish and forming some colonies by repeatedly replacing a culture medium. In this case, positions at which the colonies appear are random in the flat Petri dish and the colonies also vary in size, and hence it is difficult to estimate the number of cultured cells.

In order to solve this problem, in International Publication No. WO2015/125742, there is disclosed a cell culture vessel including a flow passage in which a plurality of depressions are formed and a culture substrate is installed at a position opposed to the depressions. This is a system in which a cell suspension is caused to flow into the flow passage, a uniform amount of cells are moved into each of the depressions, the flow passage is turned over, and a plurality of uniform amounts of cells are accordingly seeded and cultured on the culture substrate. This system enables cell groups each having approximately the same number of cells to be seeded and cultured on the culture substrate in a regular manner.

In International Publication No. WO2015/125742, it is disclosed that, at a time of cell seeding, cell groups each having approximately the same number of cells can be collectively seeded at specified positions on a culture substrate in an orderly manner. It is thereby expected that colonies each having approximately the same size are cultured at specified positions on the culture substrate after the culture in an orderly manner.

However, not all cells have the same degree of culture proliferative ability, and hence colonies may be formed in various sizes after culture. In such a case, there has been a problem that it is difficult to estimate the number of cells after culture.

### SUMMARY OF THE INVENTION

The present invention has been made in view of the above-mentioned problem. An object of the present invention is to provide a cell culture system and a cell culture method that facilitate estimation of the number of cells after culture.

The present invention adopts the following configurations. That is, there is provided a cell culture system including: a culture substrate in which a plurality of depressions are arranged and to which cells are caused to adhere; a scraper configured to detach cells adhering to a region other than the plurality of depressions on the culture substrate by sliding on the culture substrate against the region other than the plurality of depressions; and a cell removal mechanism configured to remove, from the culture substrate, the cells detached by the scraper.

In addition, according to one aspect of the present invention, there is provided a cell culture system including: a culture substrate in which a plurality of depressions are arranged and to which extracellular matrices are caused to adhere; a scraper configured to detach extracellular matrices adhering to a region other than the plurality of depressions on the culture substrate by sliding on the culture substrate against the region other than the plurality of depressions; and an extracellular matrix removal mechanism configured to remove, from the culture substrate, the extracellular matrices detached by the scraper, wherein the culture substrate is configured to cause cells to adhere to the culture substrate through intermediation of the extracellular matrices.

In addition, according to one aspect of the present invention, there is provided a cell culture method including: culturing cells by causing the cells to adhere to a culture substrate in which a plurality of depressions are arranged; detaching cells adhering to a region other than the plurality of depressions on the culture substrate by causing a scraper to slide on the culture substrate against the region other than the plurality of depressions; and removing, from the culture substrate, the cells detached by the scraper.

In addition, according to one aspect of the present invention, there is provided a cell culture method including: applying extracellular matrices to a culture substrate in which a plurality of depressions are arranged; detaching, after the extracellular matrices have been applied, extracellular matrices adhering to a region other than the plurality of depressions on the culture substrate by causing a scraper to slide on the culture substrate against the region other than the plurality of depressions; removing, from the culture substrate, the extracellular matrices detached by the scraper; and seeding cells on the culture substrate subjected to the removing, and culturing the cells.

Further features of the present invention will become apparent from the following description of exemplary embodiments with reference to the attached drawings.

### BRIEF DESCRIPTION OF THE DRAWINGS

FIG. 1A is a perspective view for illustrating an outline of an overall configuration of an example of a cell culture system according to the present invention.

FIG. 1B is a top view for illustrating an outline of a culture substrate.

FIG. 1C is a cross-sectional view for illustrating a shape of a depression.

FIG. 2A is a top view for illustrating a state of a depression exhibited when cells are seeded and then cultured in one example of the cell culture system according to the present invention.

FIG. 2B is a cross-sectional view for illustrating a state of the depression exhibited when cells are seeded and then cultured.

FIG. 2C is a top view for illustrating a state of a culture substrate and a scraper exhibited when cells are seeded and then cultured.

FIG. 2D is a cross-sectional view for illustrating a state of the depression and the scraper exhibited when cells adhering to a region other than depressions are being detached by a scraper.

FIG. 2E is a top view for illustrating a state of the culture substrate and the scraper exhibited when those cells are being detached.

FIG. 2F is a top view for illustrating a state of the depression exhibited after the cells adhering to the region other than the depression have been detached by the scraper.

FIG. 2G is a cross-sectional view for illustrating a state of the depression exhibited after those cells have been detached.

FIG. 2H is a top view for illustrating a state of the culture substrate and the scraper exhibited after those cells have been detached.

FIG. 3A is a cross-sectional view for illustrating an example of a shape of a depression.

FIG. 3B is a cross-sectional view for illustrating a depression having an inclined surface.

FIG. 3C is a cross-sectional view for illustrating a depression having a recessed curved surface.

FIG. 3D is a cross-sectional view for illustrating a culture substrate in which each region other than a depression has a protruding curved surface.

FIG. 3E is a cross-sectional view for illustrating a state in which the scraper has been pressed against the culture substrate in which each region other than the depression has a protruding curved surface.

FIG. 3F is a perspective view for illustrating depressions each shaped like a groove that is long in one direction.

FIG. 3G is a perspective view for illustrating a culture substrate including depressions each shaped like a groove that is long in one direction with each region other than the depressions having a protruding curved surface.

FIG. 4A is a schematic view for illustrating an overall configuration of a cell culture system according to a first embodiment.

FIG. 4B is a top view for illustrating a culture substrate having depressions.

FIG. 4C is a cross-sectional view of a depression.

FIG. 5 is a schematic view for illustrating how the cell culture system appears during an operation of a scraper in the first embodiment.

FIG. 6 is a schematic view for illustrating how the cell culture system appears during an operation of a cell detachment mechanism in the first embodiment.

FIG. 7A is a top view for illustrating a state of the culture substrate and the depressions in the first embodiment and illustrating a state of the culture substrate exhibited after cells have been detached by the scraper in a case in which colonies larger than the depressions have been formed after cell culture.

FIG. 7B is a top view for illustrating a state of the culture substrate exhibited after cells have been detached by the scraper in a case in which colonies that do not cover entire areas of depressions have been formed after cell culture.

FIG. 7C is a top view for illustrating a state of a depression exhibited after cells have been detached by the scraper in the case in which colonies larger than the depressions have been formed after cell culture.

FIG. 7D is a cross-sectional view for illustrating a state of a depression exhibited after cells have been detached by the scraper in the case in which colonies larger than the depressions have been formed after cell culture.

FIG. 7E is a top view for illustrating a state of a depression exhibited after cells have been detached by the scraper in the case in which colonies that do not cover the entire areas of the depressions have been formed after cell culture.

FIG. 7F is a cross-sectional view for illustrating a state of a depression exhibited after cells have been detached by the scraper in the case in which colonies that do not cover the entire areas of the depressions have been formed after cell culture.

FIG. 8A is a top view for illustrating how the cell detachment mechanism is moved above the culture substrate.

FIG. 8B is a top view for illustrating how cells adhering to a depression are detached by the cell detachment mechanism.

FIG. 9 is a schematic view for illustrating an overall configuration of a cell culture system according to a second embodiment.

FIG. 10 is a system flow chart of a cell culture system according to a third embodiment.

FIG. 11 is a block diagram for illustrating a configuration example of the cell culture system according to the present invention.

FIG. 12 is a block diagram for illustrating a configuration example of the cell culture system according to the present invention.

FIG. 13 is a block diagram for illustrating a hardware configuration example of an information processing unit according to the present invention.

FIG. 14 is a top view for illustrating a width of a scraper in the present invention in a direction perpendicular to a sliding axis of a scraper on a culture substrate and a width of a depression in the present invention in the direction perpendicular to the sliding axis of the scraper on the culture substrate.

### DESCRIPTION OF THE EMBODIMENTS

In the following description, the term “sample solutions and the like” as used in the present specification refers to a cell suspension, extracellular matrices, a culture medium, an enzyme (trypsin) for detachment, phosphate-buffered saline (PBS), and the like.

A cell culture system according to the present invention includes: a culture substrate in which a plurality of depressions are arranged and to which cells are caused to adhere; a scraper configured to detach cells adhering to a region other than the plurality of depressions on the culture substrate by sliding on the culture substrate against the region other than the plurality of depressions; and a cell removal mechanism configured to remove, from the culture substrate, the cells detached by the scraper.

Further, the cell culture system according to the present invention may be a cell culture system including: a culture substrate in which a plurality of depressions are arranged and to which extracellular matrices are caused to adhere; a scraper configured to detach extracellular matrices adhering to a region other than the plurality of depressions on the culture substrate by sliding on the culture substrate against the region other than the plurality of depressions; and an extracellular matrix removal mechanism configured to remove, from the culture substrate, the extracellular matrices detached by the scraper, wherein the culture substrate is configured to cause cells to adhere to the culture substrate through intermediation of the extracellular matrices. The extracellular matrix is a substance that serves as a so-called scaffolding for cells in cell culture, and presence of the extracellular matrix on the culture substrate enables the cells to adhere to and live at a place in which the extracellular matrix is present on the culture substrate. Examples of the extracellular matrix include collagen, vitronectin, fibronectin, and laminin.

In the following description, an adherent cell is described as an example, but the cell culture system according to the present invention can also be applied to cells other than adherent cells. Cells that are adherent and proliferative are preferred to be used, and examples thereof include ES cells, iPS cells, mesenchymal stem cells, CHO cells, and HEK293 cells.

Exemplary embodiments of the present invention are described below with reference to the accompanying drawings. In the figures, like components are denoted by like reference numerals in principle, and description thereof is omitted.

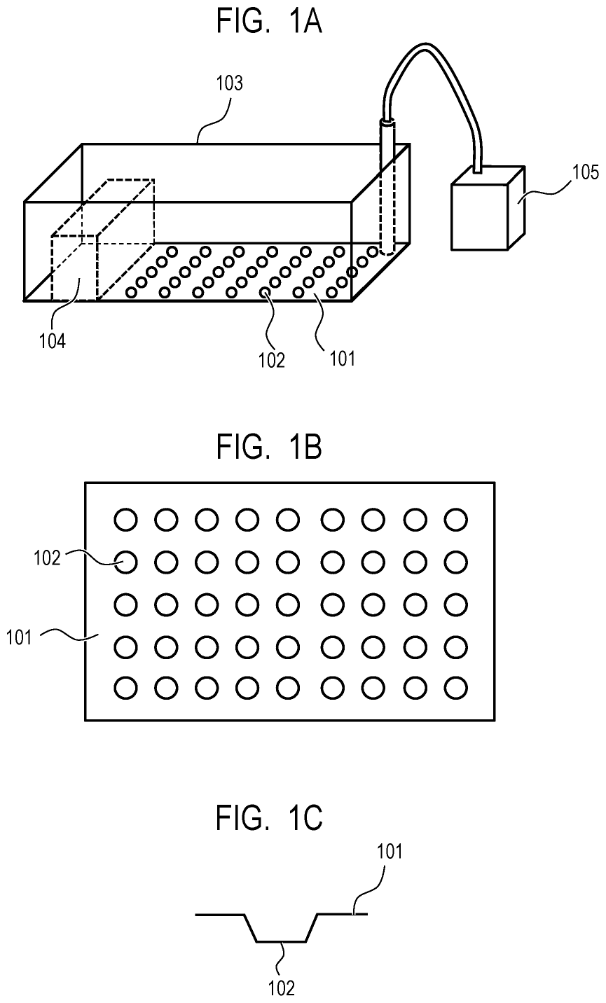

An example of the cell culture system according to the present invention is described with reference to FIG. 1A to FIG. 1C.

### (Configuration of System)

FIG. 1A is a perspective view for illustrating an outline of an overall configuration of an example of the cell culture system according to the present invention. FIG. 1B is a top view for illustrating an outline of a culture substrate. FIG. 1C is a cross-sectional view for illustrating a shape of a depression. A culture substrate 101 has a surface to which cells can adhere and on which the cells can be proliferated. On the culture substrate 101, a plurality of depressions 102, each of which has a cross section shaped as illustrated in, for example, FIG. 1C, are arranged as illustrated in FIG. 1B. A culture vessel 103 includes the culture substrate 101 and is capable of holding a liquid. A scraper 104 contacts a region other than the depressions 102 on the culture substrate 101 to detach cells adhering to the region other than the depressions 102. A cell removal mechanism 105 moves the cells detached by the scraper 104 from the culture substrate and removes the cells.

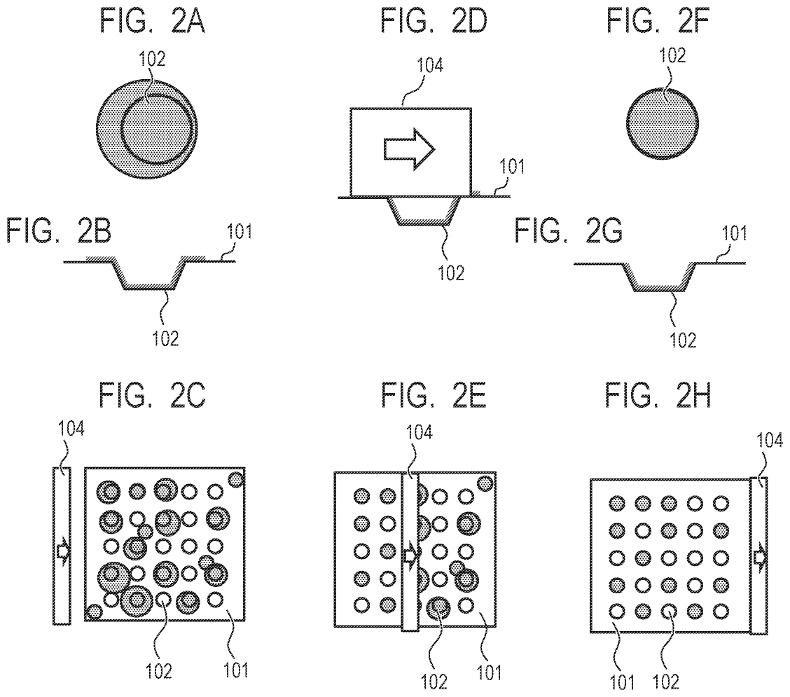

FIG. 2A to FIG. 2H are views for illustrating states before and after cells are detached by the scraper 104 in an example of the cell culture system according to the present invention. In each of FIG. 2A and FIG. 2F, the dotted portion indicates adhering cells. In each of FIG. 2C, FIG. 2E, and FIG. 2H, the dotted portions on the culture substrate 101 each indicate adhering cells. In each of FIG. 2B, FIG. 2D, and FIG. 2G, a portion indicated by the thick line of thin ink indicates adhering cells. FIG. 2A is a top view for illustrating a state of a depression 102 exhibited when cells are seeded on the culture substrate 101 and then cultured thereon. FIG. 2B is a cross-sectional view for illustrating a state of the depression 102 exhibited during culture. FIG. 2C is a top view for illustrating a state of the culture substrate 101 and the scraper 104 exhibited during culture. FIG. 2D and FIG. 2E are views for each illustrating how cells adhering to the region other than the depressions 102 on the culture substrate 101 are being detached while the scraper 104 is being moved in a direction indicated by the arrow from the above-mentioned state.

FIG. 2D is a cross-sectional view for illustrating a state of the depression 102 and the scraper 104 exhibited when cells adhering to the region other than the depression 102 are being detached by the scraper 104. FIG. 2E is a top view for illustrating a state of the culture substrate 101 and the scraper 104 exhibited when the cells are being detached. Through the detachment by the scraper 104, a colony that matches a size of each depression 102 is left on the culture substrate 101. FIG. 2F is a top view for illustrating a state of the depression 102 exhibited after the cells adhering to the region other than the depression 102 have been detached by the scraper 104. FIG. 2G is a cross-sectional view for illustrating a state of the depression 102 exhibited after the cells have been detached. FIG. 2H is a top view for illustrating a state of the culture substrate 101 and the scraper 104 exhibited after the cells have been detached. Specifying a size of the colony by the size of the depression 102 enables the number of cells present in the depression 102 to be roughly specified, and counting the number of the depressions 102 in which colonies are present enables the number of cells after culture to be easily estimated.

Now, components included in the cell culture system according to the present invention are described.

### (Culture Substrate)

The culture substrate in the present invention can be set as a culture substrate in which a plurality of depressions are arranged and to which cells are caused to adhere. The culture substrate in the present invention can also be set as a culture substrate in which a plurality of depressions are arranged and to which extracellular matrices are caused to adhere. In a case of causing the extracellular matrices to adhere to the culture substrate in the present invention, cells can be caused to adhere to the culture substrate through intermediation of the extracellular matrices.

A culture substrate is a culture surface that enables cells to adhere thereto and proliferate thereon, and a transparent material that facilitates observation is preferred to be used for the culture substrate. A transparent resin, glass, or the like that facilitates processing and observation is preferred as a material of the culture substrate. In a case of using a resin, polystyrene, polycarbonate, acrylic, or the like is usable. The culture substrate may form a part of the culture vessel, or may be provided separately from the culture vessel. In terms of cost and ease of preparation, it is preferable that the culture substrate is the same material as the culture vessel and that the culture substrate forms a part of the culture vessel.

A surface treatment such as a plasma treatment is preferred to be applied to the culture substrate so that cells are likely to adhere only to the culture substrate.

### (Culture Vessel)

The culture vessel is a container that holds a culture medium for culturing cells. A transparent resin, glass, or the like that facilitates processing and observation is preferred as a material of the culture vessel. In a case of using a resin, polystyrene, polycarbonate, acrylic, or the like is usable. The culture vessel is preferred to be equipped with a lid from the viewpoint of preventing foreign matter from entering the culture vessel from the outside and ensuring a predetermined temperature and a predetermined humidity. The culture vessel is also preferred to have a wider opening from the viewpoint of inserting a water injection and drainage port at a time of culture medium replacement and, for use of a scraper, driving the scraper. However, a closed cell culture system in which the culture vessel is set as a closed vessel can also be achieved in a case of, for example, employing a mechanism in which a tube connected to the culture vessel is used to perform water injection and drainage and the scraper is operated by gravity or magnetic force without directly touching the scraper.

### (Scraper)

The scraper in the cell culture system according to the present invention is configured to slide on the culture substrate against the region other than the depressions on the culture substrate and can detach cells adhering to the region other than the depressions.

The scraper in the cell culture system according to the present invention is configured to slide on the culture substrate against the region other than the depressions on the culture substrate and can detach extracellular matrices adhering to the region other than the depressions.

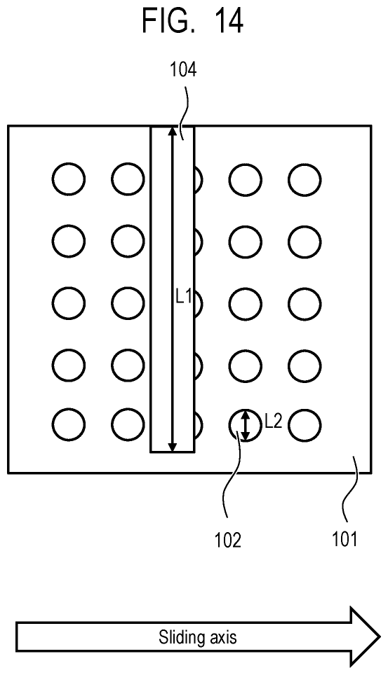

The scraper is preferred to be configured so that a surface of the scraper does not reach an inner surface of a depression during an operation of the scraper. In regard to a size of the scraper, the surface of the scraper that is brought into contact with the culture substrate is preferred to be larger than the depression in order to prevent cells or extracellular matrices in the depression from being detached. Specifically, a width of the scraper in a direction perpendicular to a sliding axis of the scraper on the culture substrate is preferred to be larger than a width of a depression in the direction perpendicular to the sliding axis of the scraper on the culture substrate. The width of the scraper in the direction perpendicular to the sliding axis of the scraper on the culture substrate is a width of the surface of the scraper that is brought into contact with the culture substrate, and refers to a maximum width of the scraper in the direction perpendicular to the sliding axis in a bottom view of the scraper. The width of the depression in the direction perpendicular to the sliding axis of the scraper on the culture substrate is a width of the depression in a top view, and refers to a maximum width in the direction perpendicular to the sliding axis of the scraper. FIG. 14 is a top view for illustrating a width L1 of the scraper 104 in the direction perpendicular to the sliding axis of the scraper 104 on the culture substrate 101 and a width L2 of the depression 102 in the direction perpendicular to the sliding axis of the scraper 104 on the culture substrate 101. With the above-mentioned configuration, it is possible to prevent the scraper from being brought into contact with cells in the depression without finely controlling the scraper by an automatic stage or the like during sliding of the scraper. That is, cells adhering to the region other than the depressions on the culture substrate can be detached by simple control for pressing the scraper against the culture substrate. The size of the scraper can be set equal to or less than a size that can accommodate the scraper in the culture vessel. A material of the scraper requires a certain degree of hardness for pressing the scraper against the culture substrate, and a metal, a resin, rubber, or the like can be used as the material of the scraper. The surface of the scraper that is brought into contact with the culture substrate is preferred to be a flat surface so that cells adhering to the region other than the depressions on the culture substrate can be efficiently detached. The scraper can be brought into contact with only the region other than the depressions on the culture substrate.

### (Depression)

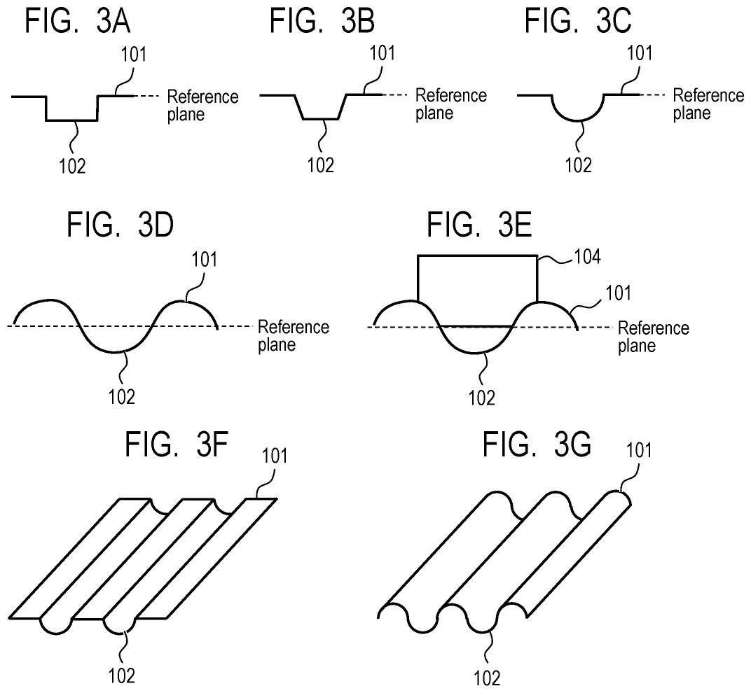

In the present invention, the depression refers to a region that is lower than a periphery thereof, and is provided on the culture substrate. That is, the depression refers to a portion that is lower than a reference plane of the culture substrate. Examples of the shape of a depression in the present invention are described with reference to FIG. 3A to FIG. 3G. In FIG. 3A to FIG. 3E, a region lower than the periphery is the depression 102, and a portion lower than a reference plane indicated by the broken line corresponds to the depression 102. When the culture substrate 101 does not have a protruding curved surface, a surface of the culture substrate 101 at a highest position can be set as the reference plane, and a surface that is a lower position than the reference plane can be set as the depression 102 (FIG. 3A to FIG. 3C). When the culture substrate 101 has a protruding curved surface, a middle level between an upper surface and a lower surface of unevenness of the culture substrate 101 can be set as the reference plane, and a surface at a lower position than the reference plane can be set as the depression 102 (FIG. 3D and FIG. 3E). The scraper is preferred to be configured to avoid being brought into contact with a region recessed from the reference plane on the culture substrate.

The shape of the depression exhibited when the culture substrate is viewed from the upper surface side is preferred to be a circle, which is similar to a shape of a colony, but may be a polygon. The depression is preferred to have an inclined surface, and can further have a recessed curved surface. The depression can be formed only of a recessed curved surface. FIG. 3A is a cross-sectional view for illustrating an example of a shape of the depression 102. FIG. 3B is a cross-sectional view for illustrating the depression 102 having an inclined surface. FIG. 3C is a cross-sectional view for illustrating the depression 102 having a recessed curved surface. As illustrated in FIG. 3B and FIG. 3C, the shape of the cross section of the depression 102 is further preferred to be inclined with a central portion of the depression 102 being recessed. When the depression has an inclined surface and has a shape recessed in the central portion, cells gather in the central portion of the depression when cells are seeded, and a colony tends to be generated from the central portion of the depression. As a result, a probability that a colony after the culture may cover the entire depression increases, and a state in which cells cover the entire depression tends to occur after the cells have been detached by the scraper.

Further, the region other than the depressions on the culture substrate can have a protruding curved surface. The region other than the depressions on the culture substrate can be formed only of a protruding curved surface. FIG. 3D is a cross-sectional view for illustrating the culture substrate 101 in which each region other than a depression 102 has a protruding curved surface. A feature of this form is that, when cells are seeded, the cells seeded in the region other than the depression on the culture substrate can roll on a protruding surface to move into the depression. Accordingly, the seeded cells can be efficiently moved into the depression. FIG. 3E is a cross-sectional view for illustrating a state in which the scraper 104 has been pressed against the culture substrate 101 having the shape illustrated in FIG. 3D. When a cushioned rubber material is used as the scraper 104, as illustrated in FIG. 3E, the scraper 104 is deformed along a protruding portion to be brought into contact with a depth to a certain extent. It is possible to detach cells on the protruding portion by moving the scraper in a horizontal direction under a state in which the scraper has been deformed along the protruding portion on the culture substrate to be brought into contact with the protruding portion. In order to detach cells on the protruding portion uniformly in the periphery, the scraper is desired to be reciprocated several times in a left-and-right direction or in a vertical and horizontal directions. A preferred degree of pressing the scraper is a degree to which cells between adjacent depressions (recessed portions) have been detached and a colony remaining in the depression has become separated from a colony in an adjacent depression.

When the cross-sectional view of the culture substrate 101 is in such a form as illustrated in FIG. 3D, cells can be efficiently moved into the depression 102 by swinging or vibrating the culture substrate 101 after the cells have been seeded, which is accordingly effective.

In a case of culturing of cells forming a colony, the size of the depression is preferred to be smaller than a colony size suitable for a passage of cells to be cultured. For example, in a case of culturing iPS cells, when the shape of the depression is a circle when viewed from the upper surface side, the depression is preferred to have such a size that the circle has a diameter of about 500 μm or less. When the shape of the depression is a polygon when viewed from the upper surface side, the depression can be set to have such a size that, for example, a diameter of a circle inscribed in the polygon, which is a shape of the depression viewed from the upper surface side, is 500 μm or less. In another case, the depression can be set to have such a size that, for example, a longest diagonal line of the polygon, which is a shape of the depression viewed from the upper surface side, is 500 μm or less. In addition, a depth of a deepest portion of the depression is preferred to be not too deep for collecting cells in the depression. For example, the depth is preferred to be about half or less of the diameter of the circle exhibited when the depression is viewed from the upper surface side, and in the case of iPS cells, is desired to be 250 μm or less.

A depression such as a groove that is long in one direction as in FIG. 3F and FIG. 3G can also be employed. FIG. 3F is a perspective view for illustrating the depressions 102 each shaped like a groove that is long in one direction. FIG. 3G is a perspective view for illustrating the culture substrate 101 including the depressions 102 each shaped like a groove that is long in one direction with each region other than the depressions 102 having a protruding curved surface. The depression shaped like a groove that is long in one direction is a depression that is particularly effective for cells that do not form a colony. It is preferred that the width of the scraper be set larger than the shorter width of each groove and the scraper be not brought into contact with the inner surface of the depression.

As illustrated in FIG. 3A, FIG. 3B, and FIG. 3C, when the region other than the depressions on the culture substrate is a flat surface, a shape and a material of the scraper are not particularly limited, and any shape and any material can be used.

The shape of the scraper that can be used in that case is, for example, a cylindrical shape, a columnar shape, or a rectangular parallelepiped. As the material of the scraper in the above-mentioned case, for example, a metal, a resin, rubber, or the like can be used.

As illustrated in FIG. 3D, when the region other than the depressions on the culture substrate has a protruding curved surface, the shape of the scraper is not particularly limited, and any shape can be used. For example, the scraper having any one of the above-mentioned shapes can be used. As the material of the scraper in the above-mentioned case, a cushioned rubber material can be used.

### (Movement of Scraper)

In order to ensure that cells or extracellular matrices are detached, the scraper is required to be securely brought into contact with the culture substrate. In that case, in order to examine contact, it is preferred to provide a contact sensor, a pressure sensor, or a system in which an image pickup apparatus such as a camera is installed to examine contact based on an image.

Further, in a case of detaching cells or extracellular matrices, the scraper is moved in a horizontal direction. This movement is carried out until the cells or the extracellular matrices adhering to a region other than the depressions have been detached. An automatic stage or the like can be used as a unit for moving the scraper. The scraper moves so as to pass through the entire region of the culture substrate at least once. This ensures that the cells or the extracellular matrices adhering to the region other than the depressions on the culture substrate are securely brought into contact with the scraper to be detached.

It is also possible to detach cells or extracellular matrices by fixing a position of the scraper and moving the culture vessel. The culture substrate may be pressed against the scraper, or the culture vessel may be moved in a horizontal direction under a pressed state. An automatic stage can be used as a unit for moving the culture vessel at this time.

### (Cell Removal Mechanism or Extracellular Matrix Removal Mechanism)

The detached cells are required to be removed from the culture vessel so that the cells do not proliferate again on the culture substrate.

The detached extracellular matrix can also be removed from the culture vessel when the culture medium is replaced. A cell removal mechanism or an extracellular matrix removal mechanism is a mechanism for removing, from the culture substrate, cells or extracellular matrices detached by the scraper, and can be an apparatus including a tank for accumulating the removed cells or extracellular matrices and an aspirator. The cell removal mechanism or the extracellular matrix removal mechanism can include a component for a tip and a tube connected to the component for a tip and the tank. The component for a tip is arranged so as to reach the culture substrate in the culture vessel. Instead of using the component for a tip, the tube may be installed so as to extend to the culture substrate. The aspirator has a mechanism in which the aspirator is activated to cause a cell suspension or extracellular matrices in a vicinity of the culture substrate to be sucked from the component for a tip or the tube and gathered in the tank. This tank can also be used for collection of an old culture medium that is discharged at a time of culture medium replacement. In addition to the aspirator, a tube pump or the like can also be used.

Further, in order to remove the detached cells or extracellular matrices, the surface of the culture substrate can be rinsed several times through use of phosphate-buffered saline (PBS) or the like. For example, after the cell suspension or the extracellular matrices have been collected, PBS is injected into the culture vessel, and the culture vessel is swung to left and right to move the PBS to the tank. This operation is repeated several times, to thereby cause a decrease in the number of detached cells or extracellular matrices in the culture vessel. Tilting the culture vessel toward the component for a tip or the tube side of the cell removal mechanism or the extracellular matrix removal mechanism has an effect of gathering the detached cells or the like on the component for a tip or the tube side, and is accordingly preferred from the viewpoint of efficiently collecting the detached cells or the like.

### (Cell Detachment Mechanism)

The cell culture system according to the present invention can include: a cell detachment mechanism for detaching cells from the depressions; and a cell collection mechanism for collecting the cells detached by the cell detachment mechanism.

The cell detachment mechanism is a mechanism for performing processing for detaching cells adhering to a depression of the culture substrate. Examples thereof include an apparatus having a nozzle for jetting sample solutions such as PBS toward the cells on the culture substrate. The cell detachment mechanism can be controlled by a cell detachment mechanism position control unit. Examples of the cell detachment mechanism position control unit include an automatic stage. The cell detachment mechanism can be moved by the cell detachment mechanism position control unit to above the depression on the culture substrate to which cells to be detached adhere, to thereby be able to perform cell detachment.

### (Cell Collection Mechanism)

The cell collection mechanism is a mechanism for collecting cells that have been detached by the cell detachment mechanism. The cell collection mechanism can be an apparatus including a tank for accumulating transferred cells and an aspirator in the same manner as the above-mentioned cell removal mechanism. The cell collection mechanism can include a component for a tip and a tube connected to the component for a tip and the tank. The component for a tip is installed so as to reach the culture substrate in the culture vessel. After the cells have been detached by the cell detachment mechanism, the aspirator of the cell collection mechanism can be activated to cause a cell suspension in the vicinity of the culture substrate to be sucked from the component for a tip and gathered in the tank. In addition to the aspirator, a tube pump or the like can also be used, and instead of using the component for a tip, the tube may be installed so as to extend to the culture substrate side. It is preferred that, after the cells have been collected, PBS, a culture medium, or the like be injected from a liquid feeding mechanism and the cells that have failed to be collected be collected again by the cell collection mechanism.

### (Image Pickup Apparatus)

The cell culture system according to the present invention can include an image pickup apparatus that allows observation of a state of cells. The cell culture system according to the present invention can include an image pickup apparatus that allows observation of a detachment residue of cells adhering to the region other than the depressions. The image pickup apparatus can also acquire, for example, information regarding an adhesion status of cells or extracellular matrices on the culture substrate. Examples of the image pickup apparatus include a CMOS camera and a CCD camera. It is also effective to install the image pickup apparatus, determine a detachment status of the cells from an image acquired by the image pickup apparatus, and move the scraper until all the cells except those in the depressions have been detached. It is also effective to detect the cells that remain undetached from an image acquired by the image pickup apparatus and move the scraper or the like to that place. It is preferred that a state of cells in each depression be determined based on information acquired by the image pickup apparatus, a depression to which the cells to be detached adhere be selected based on the state of cells, the cells be detached from the selected depression by the cell detachment mechanism, and the cells detached be collected by the cell collection mechanism. As the image pickup apparatus in the present invention, a single apparatus may be used, or a plurality of apparatus may be used.

### (Information Processing Unit)

The cell culture system according to the present invention can include an information processing unit. The information processing unit in the present invention can include: a cell determination function of determining a state of cells in each of the depressions based on the state of cells acquired by the image pickup apparatus; and a depression selection function of selecting a depression to which the cells to be detached adhere based on the state of cells in each of the depressions which has been determined by the cell determination function. The information processing unit in the present invention can be an information processing unit configured to operate, when the detachment residue is identified, the scraper so as to remove the detachment residue. The information processing unit can process, for example, information regarding the state of cells on the culture substrate, in particular, for example, information regarding the state of cells on the culture substrate which have been acquired by the image pickup apparatus. The information processing unit can use the cell determination function to determine the state of cells in each depression based on the information acquired by the image pickup apparatus. The cell determination is preferred to be performed based on the area of cells that cover each depression. It is also effective to use, for the determination, a difference in output value of an image. A change occurs in output value of an image at a cell position depending on a degree of overlap and a density of cells. In view of this, a specified output value is determined in advance, and it is determined whether or not an image output value of a depression in which cells are positioned corresponds to the specified output value, and a colony in a state close to an assumed state can thereby be selected, thereby improving estimation accuracy of the number of cells. Then, the depression selection function enables a depression from which cells are to be collected to be selected based on the state of cells which has been determined by the cell determination function. Those processes can be achieved through use of a PC, various microcomputers, and the like that are described later.

The information processing unit can also determine whether or not a desired amount of cells have been cultured based on, for example, information on the areas of colonies included in the culture substrate which has been acquired by the image pickup apparatus, and issue an instruction to continue the culture or an instruction to end the culture and shift to the cell detachment by the scraper. In a case of cells having a stable proliferation rate, the information processing unit can also be used to set in advance the cell detachment to start after a lapse of a specified culture time period, and issue an instruction to perform the cell detachment after the lapse of the culture time period. In addition, for example, it is possible to issue an instruction to perform processing for detaching cells in a desired region by examining an adhesion region of cells from an image acquired by the image pickup apparatus and moving the scraper to the adhesion region.

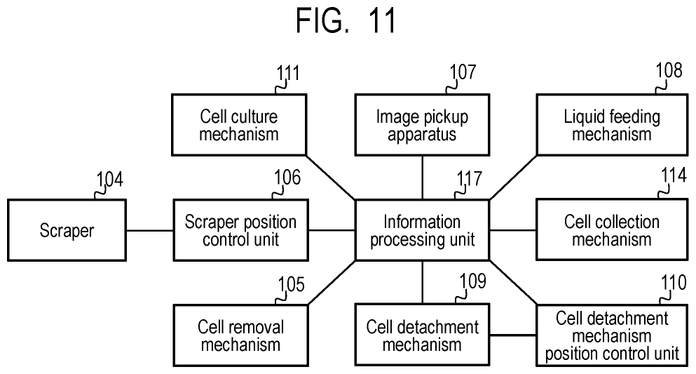

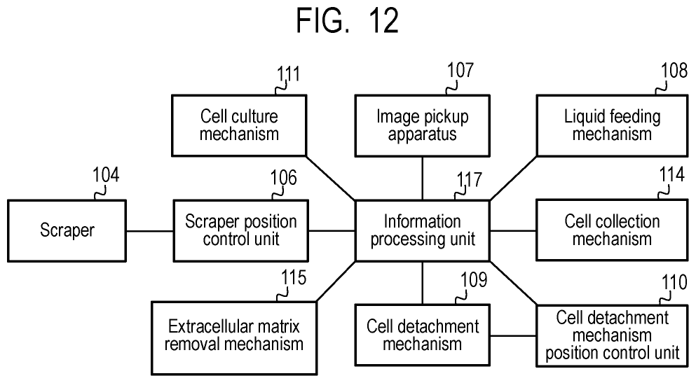

FIG. 11 is a block diagram for illustrating a configuration example of the cell culture system according to the present invention. The cell removal mechanism 105 can be an extracellular matrix removal mechanism 115 as illustrated in FIG. 12. In a case of using the cell culture system having such a configuration as illustrated in FIG. 11 or FIG. 12, an information processing unit 117 can, for example, instruct a cell culture mechanism 111 to perform processing for performing the cell culture for a desired time period. Further, for example, a liquid feeding mechanism 108 can be instructed to inject a desired amount of sample solutions and the like at a desired timing. Further, for example, it is possible to control a scraper position control unit 106 for moving the scraper 104 onto the culture substrate 101 at a time of the cell detachment. Further, for example, the cell removal mechanism 105 or the extracellular matrix removal mechanism 115 can be instructed to perform processing for removing sample solution such as cells, extracellular matrices, and a culture medium at a desired timing. Further, for example, a cell detachment mechanism 109 and a cell detachment mechanism position control unit 110 can be instructed to perform processing for the cell detachment at a desired timing. The above-mentioned control of the scraper position control unit 106 or the above-mentioned control of the cell detachment mechanism 109 and the cell detachment mechanism position control unit 110 can be performed after the information processing unit 117 has processed the information regarding the state of cells which has been acquired from an image pickup apparatus 107. Further, for example, a cell collection mechanism 114 can be instructed to perform processing for collecting cells at a desired timing.

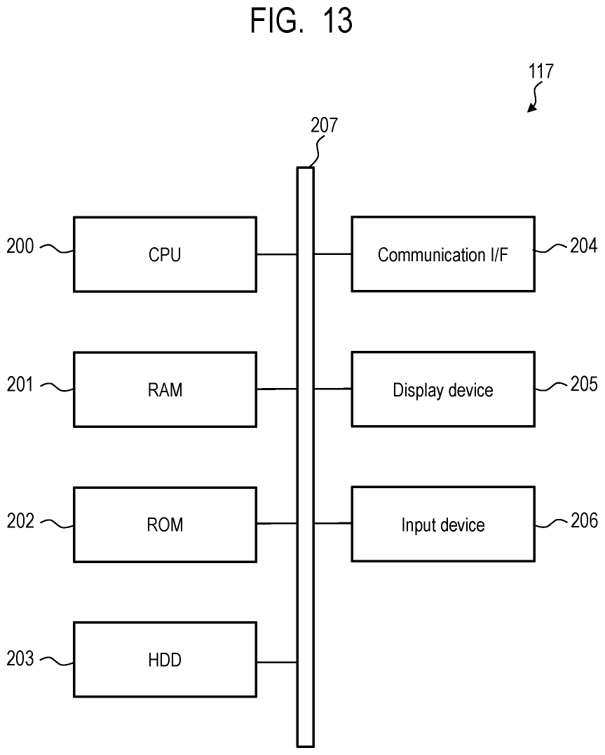

FIG. 13 is a block diagram for illustrating a hardware configuration example of an information processing unit 117 in the present invention. The information processing unit 117 has functions of a computer. For example, the information processing unit 117 may be configured unitarily with a desktop personal computer (PC), a laptop PC, a tablet PC, or a smartphone, for example. The information processing unit 117 includes, in order to implement functions as a computer that performs arithmetic operation and storage, a central processing unit (CPU) 200, a random-access memory (RAM) 201, a read-only memory (ROM) 202, and a hard disk drive (HDD) 203. The information processing unit 117 also includes a communication interface (I/F) 204, a display device 205, and an input device 206. The CPU 200, the RAM 201, the ROM 202, the HDD 203, the communication I/F 204, the display device 205, and the input device 206 are connected to each other via a bus 207. The display device 205 and the input device 206 may be connected to the bus 207 via a drive device (not shown) for driving those devices.

In FIG. 13, the various components forming the information processing unit 117 are illustrated as an integrated device, but some of the functions of those components may be implemented by an external device. For example, the display device 205 and the input device 206 may be external devices different from the components implementing the functions of the computer including the CPU 200, for example.

The CPU 200 performs predetermined operations in accordance with programs stored in, for example, the RAM 201 and the HDD 203, and also has a function of controlling each component of the information processing unit 117. The RAM 201 is built from a volatile storage medium, and provides a temporary memory area required for the operations of the CPU 200. The ROM 202 is built from a non-volatile storage medium, and stores required information such as programs to be used for the operations of the information processing unit 117. The HDD 203 is formed of a nonvolatile storage medium, and is a storage device that stores coordinates indicating a position on the culture substrate, an amount of cells, information regarding the state of cells, and the like.

The communication I/F 204 is a communication interface based on a standard such as Wi-Fi (trademark) or 4G, and is a module for communicating to and from another device. The display device 205 is, for example, a liquid crystal display or an organic light emitting diode (OLED) display, and is used for displaying moving images, still images, and characters, for example. The input device 206 is, for example, a button, a touch panel, a keyboard, or a pointing device, and is used by a user to operate the information processing unit 117. The display device 205 and the input device 206 may be integrally formed as a touch panel.

The hardware configuration illustrated in FIG. 13 is an example, and devices other than the illustrated devices may be added, or some of the illustrated devices may be omitted. Further, some of the devices may be substituted with another device having the same function. Moreover, some of the functions may be provided by another device via a network, and the functions for implementing the embodiments may be shared and implemented by a plurality of devices. For example, the HDD 203 may be substituted with a solid state drive (SSD) which uses a semiconductor element, such as a flash memory, or may be substituted with cloud storage.

The CPU 200 implements the cell determination function and the depression selection function by loading a program stored in the ROM 202 or the like into the RAM 201 and executing the program. The CPU 200 also controls the display device 205. The CPU 200 also controls the HDD 203.

### (Cell Culture Mechanism)

The cell culture system according to the present invention can include a cell culture mechanism configured to culture cells. Examples of the cell culture mechanism include a heat retaining unit that maintains the entire culture vessel at 37° C., a unit that maintains the entire culture vessel under a CO₂ atmosphere, and a unit that maintains the entire culture vessel at a humidity of 95%. Specifically, an incubator in which the entire culture vessel is maintained at a temperature of 37° C., a CO₂ concentration of 5%, and a humidity of 95% is given as an example. In the following embodiments, cells are seeded by a liquid feeding mechanism using a tube and cultured, but cells may be seeded through use of a pipette.

Through use of the above-mentioned system, places in which colonies are cultured can be limited to only within depressions each having a specified size. Accordingly, it is possible to provide a cell culture system that facilitates estimation of the number of cells present on a culture substrate after cell culture.

### (Cell Culture Method)

A cell culture method according to the present invention can include: culturing cells by causing the cells to adhere to a culture substrate in which a plurality of depressions are arranged; detaching cells adhering to a region other than the plurality of depressions on the culture substrate by causing a scraper to slide on the culture substrate against the region other than the plurality of depressions; and removing, from the culture substrate, the cells detached by the scraper.

Further, a cell culture method according to the present invention can include: applying extracellular matrices to a culture substrate in which a plurality of depressions are arranged; detaching, after the extracellular matrices have been applied, extracellular matrices adhering to a region other than the plurality of depressions on the culture substrate by causing a scraper to slide on the culture substrate against the region other than the plurality of depressions; removing, from the culture substrate, the extracellular matrices detached by the scraper; and seeding cells on the culture substrate subjected to the removing, and culturing the cells.

### First Embodiment

Now, the configuration of the cell culture system according to a first embodiment and the cell culture method are described. In the figures, like components are denoted by like reference numerals in principle, and description thereof is omitted.

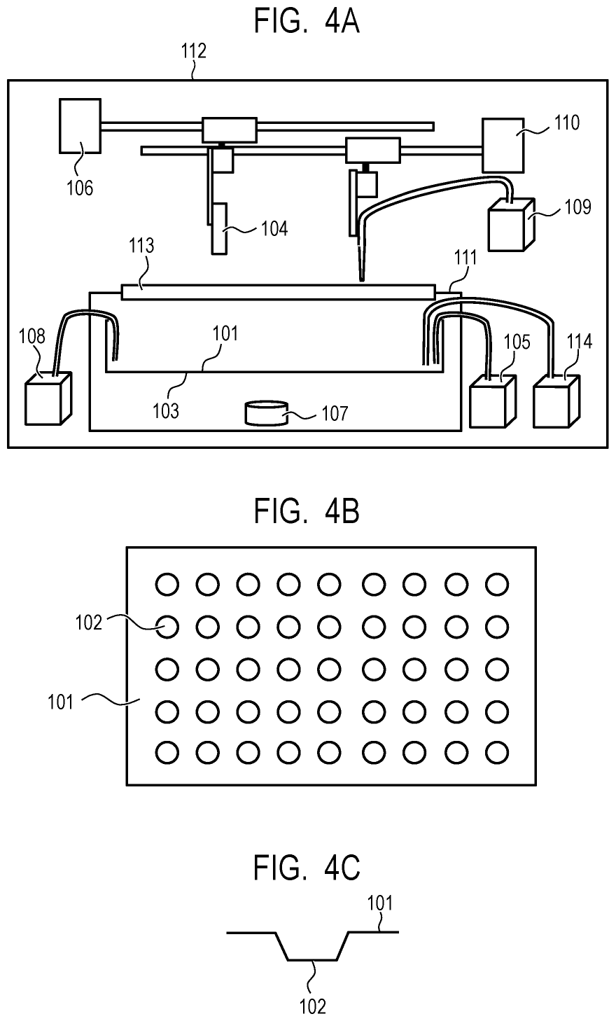

FIG. 4A is a schematic view for illustrating an overall configuration of the cell culture system according to the first embodiment. FIG. 4B is a top view for illustrating the culture substrate 101 having the depressions 102. FIG. 4C is a cross-sectional view of the depression 102. This cell culture system includes the culture vessel 103 made of polystyrene having the culture substrate 101 on a bottom surface thereof having a plurality of depressions 102 each having a diameter of @0.5 mm and a depth of 0.2 mm as illustrated in FIG. 4B and FIG. 4C. The scraper 104 made of rubber and the scraper position control unit 106 for controlling the position of the scraper are provided above the culture vessel 103. The scraper position control unit 106 is formed of a combination of a two-axis stage that moves in a horizontal direction with respect to the culture substrate 101 and a one-axis stage that moves in a vertical direction, and is provided with a contact sensor (not shown) that detects contact between the scraper 104 and the culture vessel 103.

On side surfaces of the culture vessel 103, a plurality of tubes are arranged along wall surfaces, and one of the tubes is connected to the cell removal mechanism 105 for collecting the cells detached by the scraper 104. The cell removal mechanism 105 is formed of a plastic tank and an aspirator (not shown), and has a function of collecting waste liquid such as a used culture medium as well as the detached cells. Some other tubes are connected to the liquid feeding mechanism 108 that feeds the sample solutions and the like to the culture vessel 103. The liquid feeding mechanism 108 is formed of plastic containers in which the respective sample solutions and the like are packaged and a tube pump. In the first embodiment, four containers in which a cell suspension, a culture medium, an enzyme (trypsin) for detachment, and PBS are contained as the sample solutions and the like are prepared. In FIG. 4A to FIG. 4C, only one of the containers containing the sample solutions and the like is illustrated as the liquid feeding mechanism 108, and the tube pump is not shown. In addition, one of the tubes is connected to the cell collection mechanism 114 that eventually collects required cells after the required cells have been detached. The cell collection mechanism 114 is formed of a plastic container and an aspirator (not shown).

A CMOS camera is installed in a central portion below the culture vessel 103 as the image pickup apparatus 107 that allows observation of the culture substrate 101. A CCD camera can also be used therefor instead. An incubator is also installed as the cell culture mechanism 111. The culture vessel 103 and the image pickup apparatus 107 are installed in the cell culture mechanism 111, and the inside of the culture vessel 103 is always maintained at a CO₂ concentration of 5%, a humidity of 95%, and a temperature of 37° C. In an upper portion of the cell culture mechanism 111, a lid 113 of which opening and closing can be automatically controlled is present so as to allow the scraper 104 and the like to be taken in and out of the culture vessel 103. Above the lid 113, the cell detachment mechanism 109 for detaching required cells after culture through use of a water flow and the cell detachment mechanism position control unit 110 for controlling a position of the cell detachment mechanism 109 are arranged. The cell detachment mechanism position control unit 110 is formed of a combination of a two-axis stage that moves in a horizontal direction with respect to the culture substrate 101, and a one-axis stage that moves in a vertical direction with respect to the culture substrate 101.

The above-mentioned components are installed in a safety cabinet 112 so that the cell culture can be achieved in a clean region.

A procedure of actual cell culture using this system is described below.

### (Cell Culture)

First, a cell suspension obtained by mixing a culture medium, extracellular matrices, and cells to be cultured is fed to the culture vessel 103 through use of the liquid feeding mechanism 108. The cell suspension is fed to an extent that the cell suspension is immersed over the entire area of the culture substrate 101. After that, the cell culture mechanism 111 is used to maintain the inside of the culture vessel 103 under a state in which the CO₂ concentration is 5%, the humidity is 95%, and the temperature is 37° C. The culture medium replacement is sometimes carried out through use of the liquid feeding mechanism 108 and the cell removal mechanism 105. A frequency of the culture medium replacement is preferred to be set in accordance with the cells to be cultured. Those operations are continued until the cells have been proliferated by a desired amount. Whether or not a desired amount of cells have been cultured can be determined from the area of a colony included in the culture substrate 101 through use of the image pickup apparatus 107. For example, one of criteria is whether or not a colony has grown until the colony covers each depression 102. This determination may be made by any one of automatic processing using image processing by the information processing unit (not shown) or a method in which the determination is visually made by an operator.

### (Cell Detachment by Scraper)

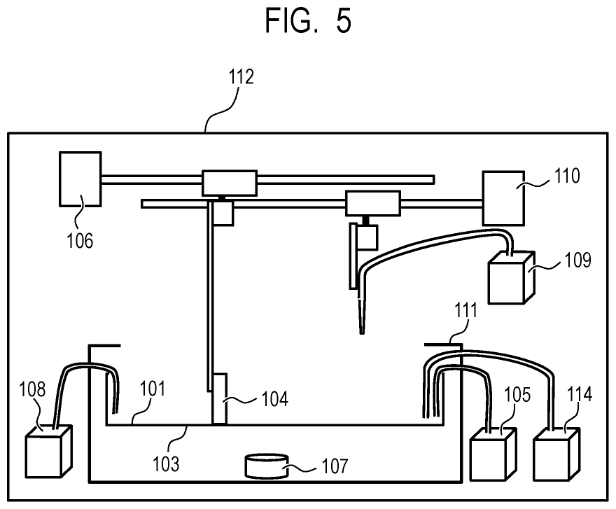

After a desired amount of cells have been cultured, the lid 113 is opened, and the scraper position control unit 106 is used to insert the scraper 104 into the culture vessel 103 and move the scraper 104 into contact with the culture substrate 101. After it is confirmed that the scraper 104 has been brought into contact with the culture substrate 101, the culture substrate 101 is scraped by the scraper 104 to detach the cells adhering to the region other than the depressions 102. FIG. 5 is a schematic view for illustrating how the cell culture system appears while the scraper 104 is operating on the culture substrate 101 in the first embodiment. After that, the image pickup apparatus 107 examines the detachment status, and when there is a detachment residue in the region other than the depressions 102 on the culture substrate 101, the culture substrate 101 is scraped again by the scraper 104. This operation causes colonies to remain only in the depressions 102. After that, the lid 113 of the cell culture mechanism 111 is closed.

The culture medium containing the detached cells is collected by the cell removal mechanism 105. After that, PBS is injected from the liquid feeding mechanism 108 to entrain the cells that have failed to be collected, and the collection is performed again by the cell removal mechanism 105. The number of rinsing operations may be increased as required.

### (Selection of Cells)

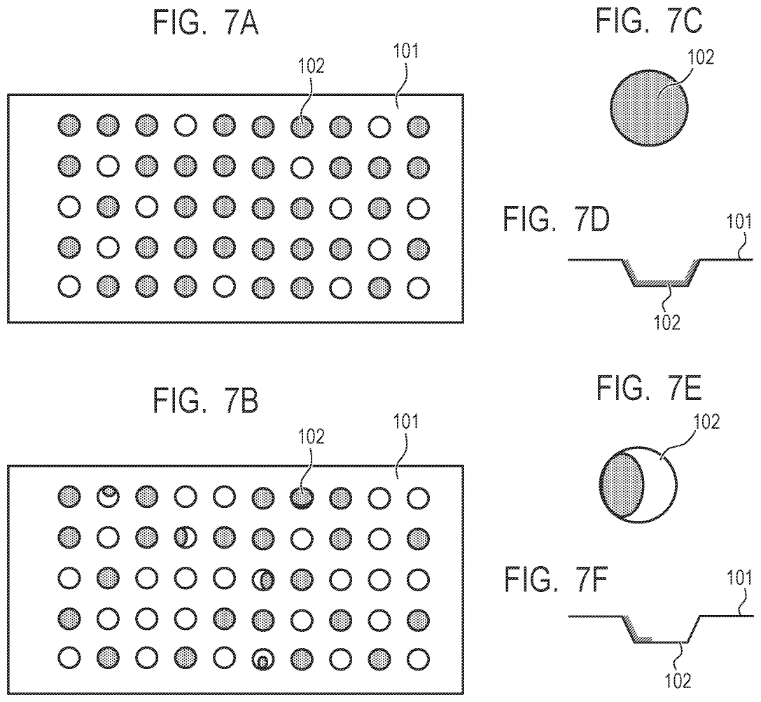

FIG. 7A to FIG. 7F are views for illustrating a state of the culture substrate 101 and the depression 102 after cell culture in the first embodiment. In each of FIG. 7A to FIG. 7F, the dotted portion indicates a portion to which cells adhere. FIG. 7A is a top view for illustrating a state of the culture substrate 101 exhibited after cells have been detached by the scraper 104 in a case in which colonies larger than the depressions 102 have been formed after cell culture. FIG. 7C is a top view for illustrating a state of a depression 102 exhibited after cells have been detached in the above-mentioned case. FIG. 7D is a cross-sectional view for illustrating a state of a depression 102 exhibited after cells have been detached in the above-mentioned case. When the culture substrate 101 has been filled with colonies larger than the depressions 102 through sufficient culture, the culture substrate 101 that has been processed by the scraper 104 appears as illustrated in FIG. 7A. At this time, colonies in each of which the entire depression 102 is covered with cells as illustrated in FIG. 7C and FIG. 7D are formed. Under this state, the image pickup apparatus 107 acquires an image of the culture substrate 101, determines each depression 102 covered with cells, and records coordinates thereof. After that, each depression 102 from which cells are to be collected is selected in accordance with the number of required cells. FIG. 7B is a top view for illustrating a state of the culture substrate 101 exhibited after cells have been detached by the scraper 104 in a case in which colonies that do not cover entire areas of depressions 102 have been formed after culture. FIG. 7E is a top view for illustrating a state of a depression 102 exhibited after the cell detachment. FIG. 7F is a cross-sectional view for illustrating a state of a depression 102 exhibited after the cell detachment. The culture substrate 101 that has been subjected to cell detachment processing by the scraper 104 under a state in which the culture is not sufficient appears as illustrated in FIG. 7B, thereby causing colonies in each of which cells do not cover the entire area of the depression 102 as illustrated in FIG. 7E and FIG. 7F. In such a case, it is preferred to determine that the amount of cells is insufficient in such a depression and to take the determination into consideration in conversion of the amount of cells to be collected.

The determination of cells in the depressions 102 based on those manners in which the depressions 102 are covered with cells may be manually performed, or may be automatically processed by the information processing unit. The automatic processing is carried out by the cell determination function included in the information processing unit.

In addition, the selection of a depression from which cells are to be collected based on a result of the cell determination may be manually performed, or may be automatically processed. The automatic processing is carried out by the depression selection function included in the information processing unit. Those processes are achieved through use of a PC, various microcomputers, and the like.

### (Detachment and Collection of Cells)

After the depression 102 from which cells are to be collected is selected, trypsin is injected into the culture vessel 103 through the liquid feeding mechanism 108. The cell culture mechanism 111 then keeps the culture substrate 101 warm under a state in which the culture substrate 101 is immersed in the trypsin. Accordingly, an adhesion force of cells can be lowered.

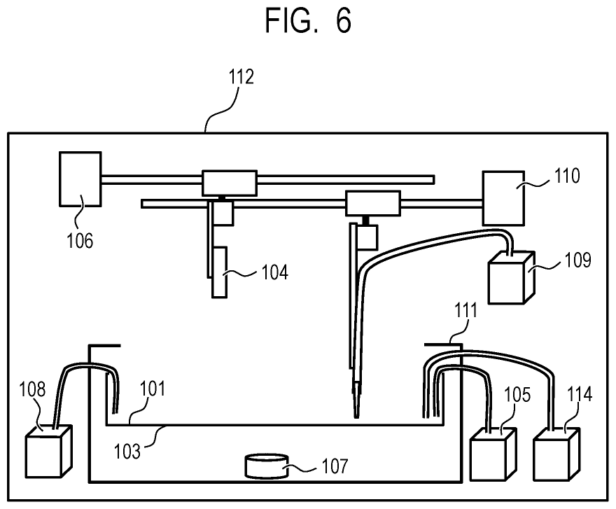

After the adhesion force of the cells is lowered, the lid 113 of the cell culture mechanism 111 is opened again, and the cell detachment mechanism 109 is inserted therethrough. FIG. 6 is a view for illustrating how the cell culture system appears during an operation of the cell detachment mechanism 109 in the first embodiment. In the cell detachment mechanism 109, a silicone tube connected to a straight jet nozzle made of a resin is connected to an arm so that a tip of the straight jet nozzle can be moved to any position. The cell detachment mechanism 109 is controlled through use of the cell detachment mechanism position control unit 110.

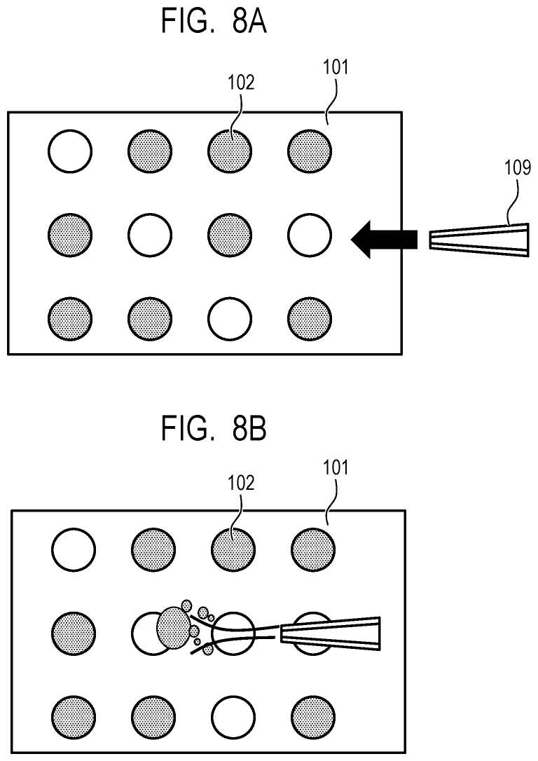

A situation of detachment is described with reference to FIG. 8A and FIG. 8B. In FIG. 8A and FIG. 8B, the dotted portion indicates a portion to which cells adhere. FIG. 8A is a top view for illustrating how the cell detachment mechanism 109 is moved above the culture substrate 101 for the cell detachment. FIG. 8B is a top view for illustrating how cells adhering to a depression 102 are detached by the cell detachment mechanism 109. The straight jet nozzle is moved in a direction indicated by the arrow to the recorded coordinates of the depression 102 as illustrated in FIG. 8A. After that, PBS is jetted from the straight jet nozzle of the cell detachment mechanism 109 to a target depression 102, and the cells in the depression 102 are detached as illustrated in FIG. 8B. This step is carried out until all the cells in the selected depression 102 have been detached.

After the colonies in all the selected depressions 102 have been detached, the cell suspension in the culture vessel 103 is collected by the cell collection mechanism 114. It is preferred that, after the cells have been collected, PBS, the culture medium, or the like be injected from the liquid feeding mechanism and the cells that have failed to be collected be collected again by the cell collection mechanism 114.

As described above, in the first embodiment, there is provided a cell culture system including: a culture substrate in which a plurality of depressions are arranged and to which cells are caused to adhere; a scraper configured to detach cells adhering to a region other than the plurality of depressions on the culture substrate by sliding on the culture substrate against the region other than the plurality of depressions; and a cell removal mechanism configured to remove, from the culture substrate, the cells detached by the scraper. Accordingly, it is possible to provide a cell culture system that facilitates estimation of the number of cells present on a culture substrate after cell culture and facilitates collection of the cells. Further, in the first embodiment, there is provided a cell culture method including: culturing cells by causing the cells to adhere to a culture substrate in which a plurality of depressions are arranged; detaching cells adhering to a region other than the plurality of depressions on the culture substrate by causing a scraper to slide on the culture substrate against the region other than the plurality of depressions; and removing, from the culture substrate, the cells detached by the scraper.

The first embodiment has been verified through use of iPS cells, but most types of cell can be used as long as the cells are adherent cells. In that case, types of culture medium and reagent and a timing of the detachment may be changed as appropriate in accordance with characteristics of each cell and a protocol to be implemented.

### Second Embodiment

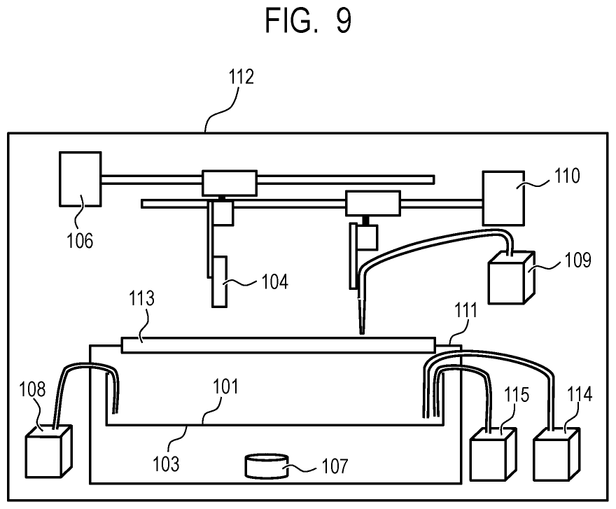

Now, the configuration of the cell culture system according to a second embodiment and the cell culture method are described with reference to FIG. 9. In FIG. 9, like components are denoted by like reference numerals in principle, and description thereof is omitted.

The cell culture system illustrated in FIG. 9 merely has the cell removal mechanism 105 in the first embodiment replaced by the extracellular matrix removal mechanism 115, and the same modules are also employed. That is, the components are the same, and only a processing procedure differs.

A configuration of the cell culture system according to the second embodiment is the same except for the extracellular matrix removal mechanism 115 described above, and hence description thereof is omitted. Instead, the cell culture method using the cell culture system is described. In this cell culture method, description of steps that are the same as the steps in the cell culture method according to the first embodiment is omitted.

### (Application of Extracellular Matrix)

First, a mixed solution of PBS and extracellular matrices is fed to the culture vessel 103 through use of the liquid feeding mechanism 108. The mixed solution is fed to an extent that the mixed solution is immersed over the entire area of the culture substrate 101, to thereby be applied to the culture substrate 101. After that, the cell culture mechanism 111 is used to maintain the culture vessel 103 under a state in which the humidity is 95%, and the temperature is 37° C. and store the culture vessel 103. After a lapse of a certain period of time, the extracellular matrix removal mechanism 115 is used to collect the mixed solution, and the liquid feeding mechanism 108 is used to inject PBS again. After that, the lid 113 of the cell culture mechanism 111 is opened, and the scraper 104 is inserted into the culture vessel 103 therethrough. After it is confirmed that the scraper 104 has been brought into contact with the culture substrate 101, the scraper 104 is brought into contact with the region other than the depressions 102 on the culture substrate 101, and the extracellular matrices adhering to the region other than the depressions 102 are detached through sliding and scraping. After that, the extracellular matrix removal mechanism 115 is used to separate liquid in the culture vessel 103, to thereby remove the detached extracellular matrices. Then, the lid 113 of the cell culture mechanism 111 is closed.

### (Cell Culture)

First, a cell suspension obtained by mixing cells to be cultured and a culture medium is fed to the culture vessel 103 through use of the liquid feeding mechanism 108, and the cells are seeded. The cell suspension is fed to an extent that the cell suspension is immersed over the entire area of the culture substrate 101. After that, the cell culture mechanism 111 is used to maintain the culture vessel 103 under a state in which the CO₂ concentration is 5%, the humidity is 95%, and the temperature is 37° C. and store the culture vessel 103. The culture medium replacement is sometimes carried out through use of the liquid feeding mechanism 108 and the extracellular matrix removal mechanism 115. A frequency of this culture medium replacement is preferred to be set in accordance with the cells to be cultured. As this operation is continued, in the same manner as in the states illustrated in FIG. 7A, FIG. 7C, and FIG. 7D, the cells are brought into a state of being adhered only to the inside of the depressions 102 in which the extracellular matrices remain. In this case, when cells adhere to the region other than the depressions 102, it is also effective to add processing for scraping off cells except those in the depressions 102 by the scraper 104 as described in the section “(Cell Detachment by Scraper)” of the first embodiment. In this case, the same mechanism can be used as the extracellular matrix removal mechanism 115 and the cell removal mechanism 105.

The subsequent steps are the same as the steps of the section “(Selection of Cells)” and subsequent steps described in the first embodiment.

As described above, in the second embodiment, there is provided a cell culture system including: a culture substrate in which a plurality of depressions are arranged and to which extracellular matrices are caused to adhere; a scraper configured to detach extracellular matrices adhering to a region other than the plurality of depressions on the culture substrate by sliding on the culture substrate against the region other than the plurality of depressions; and an extracellular matrix removal mechanism configured to remove, from the culture substrate, the extracellular matrices detached by the scraper, wherein the culture substrate is configured to cause cells to adhere to the culture substrate through intermediation of the extracellular matrices. Accordingly, it is possible to provide a cell culture system that facilitates estimation of the number of cells present on the culture substrate after cell culture and facilitates collection of the cells. Further, in the second embodiment, there is provided a cell culture method including: applying extracellular matrices to a culture substrate in which a plurality of depressions are arranged; detaching, after the extracellular matrices have been applied, extracellular matrices adhering to a region other than the plurality of depressions on the culture substrate by causing a scraper to slide on the culture substrate against the region other than the plurality of depressions; removing, from the culture substrate, the extracellular matrices detached by the scraper; and seeding cells on the culture substrate subjected to the removing, and culturing the cells.

The second embodiment has been verified through use of iPS cells, but most types of cell can be used as long as the cells are adherent cells. In that case, types of culture medium and reagent and a timing of the detachment may be changed as appropriate in accordance with characteristics of each cell and a protocol to be implemented.

### Third Embodiment

Now, the configuration of the cell culture system according to a third embodiment and the cell culture method are described. The cell culture system according to the third embodiment is an automated cell culture system. In the figures, like components are denoted by like reference numerals in principle, and descriptions thereof are omitted.

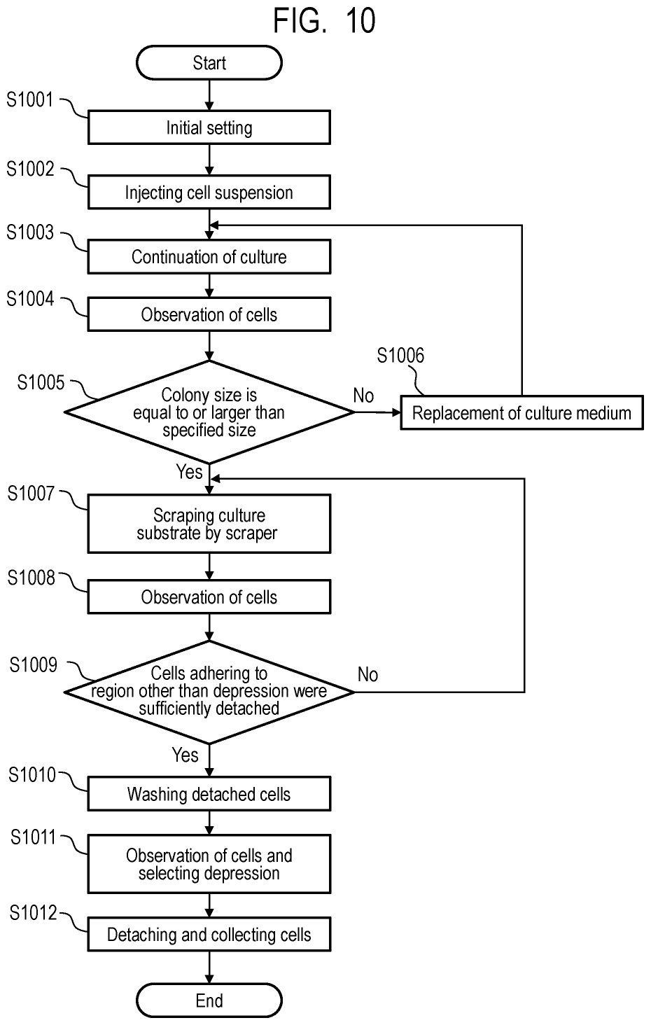

FIG. 10 is a flow chart for illustrating a system flow of the third embodiment.

A configuration of the cell culture system according to the third embodiment can be set to be the same as the configuration of the cell culture system according to the first embodiment illustrated in FIG. 4A to FIG. 4C except for the information processing unit, and hence description thereof is accordingly omitted. A main apparatus configuration of the cell culture system according to the third embodiment can be set to be the same as the configuration illustrated in FIG. 11.

Step S1001 of the following flow is carried out by manipulation, and Step S1002 and the subsequent steps are automatically operated. The automated operation of those steps is controlled by an algorithm recorded in advance in the information processing unit.

### (Manipulation Step)

(Step S1001) As an initial setting, it is examined that the environment in the cell culture mechanism 111 in the safety cabinet 112 is a CO₂ concentration of 5%, a humidity of 95%, and a temperature of 37° C. After the examination, a culture vessel 103 to which each tube is connected is installed at a predetermined position in the cell culture mechanism 111, and the liquid feeding mechanism 108 containing various sample solutions and the like and the cell suspension is set in the safety cabinet 112. After that, the amount of cells to be cultured and collected is input and set, and a setting for starting automated steps is performed.

### (Automated Steps)

(Step S1002) First, a cell suspension obtained by mixing a culture medium, extracellular matrices, and cells to be cultured is injected into the culture vessel 103 by the liquid feeding mechanism 108. The image pickup apparatus 107 is used to observe the culture substrate 101, and feeding of the cell suspension is continued until the culture substrate 101 is immersed in the cell suspension. A liquid amount by which the culture substrate 101 is immersed is confirmed in advance, and a cell suspension in which cells of an amount desired to be seeded on the culture medium or the like are mixed in advance is prepared in accordance with the liquid amount.

(Step S1003) After the cells have been seeded, the culture is continued for storage in the cell culture mechanism 111 for a certain period of time.

This storage period is preferred to be changed depending on the type of cell.

(Step S1004) The state of colonies of cells on the culture substrate 101 is observed and examined through use of the image pickup apparatus 107.

(Step S1005) It is determined whether or not the colony size is equal to or larger than a specified size. The specified size of a colony is a size set based on the area of the depression 102, and is preferred to have a size larger than the area of the depression 102 by several tens of percent or more.

(Step S1006) When the colony size is smaller than the specified size, the cell removal mechanism 105 is used to drain the culture medium in the culture vessel 103, and a new culture medium is injected from the liquid feeding mechanism 108 for replacement. After that, the steps from (Step S1003) in which the culture is continued are repeated.

(Step S1007) When the colony size is larger than the specified size, the lid 113 of the cell culture mechanism 111 is opened, and the scraper position control unit 106 is used to move the scraper 104 and detach the colonies adhering to the region other than the depressions 102 on the culture substrate 101.

(Step S1008) The image pickup apparatus 107 is used to observe the culture substrate 101.

(Step S1009) It is determined whether or not there are colonies remaining in the region other than the depressions 102 on the culture substrate 101 in the step of (Step S1008). When there are colonies remaining in the region other than the depressions 102, the steps from (Step S1007) are performed again to carry out the step of detaching the colonies on the culture substrate 101 again by the scraper 104.

(Step S1010) When it has been confirmed that the colonies in the region other than the depressions 102 on the culture substrate 101 have been detached, washing of the detached cells is executed. At this time, injection of PBS from the liquid feeding mechanism 108 and drainage by the cell removal mechanism 105 are repeated to discharge the detached cells from the culture vessel 103. After that, PBS is injected from the liquid feeding mechanism 108.

(Step S1011) The image pickup apparatus 107 is used to observe the culture substrate 101 and examine a status of cells remaining in the depressions 102. The state of the cells is determined by the cell determination function. Appropriate depressions 102 are automatically selected by the depression selection function based on a cell collection amount set in advance.

(Step S1012) After the depression 102 from which cells are to be detached is selected, the cell removal mechanism 105 is used to drain the PBS and inject trypsin for detachment into the culture vessel 103 from the liquid feeding mechanism 108 to an extent that the culture substrate 101 is immersed. After that, the adhesion force of the cells is lowered by maintaining the state of 37° C. for a certain period of time. After that, the lid 113 of the cell culture mechanism 111 is opened, and the cell detachment mechanism position control unit 110 is used to move a nozzle tip of the cell detachment mechanism 109 to the selected depression 102 and spray the PBS from the nozzle, to thereby detach the cells in the depression 102. After the cells in all the selected depressions 102 have been detached in this manner, the cell collection mechanism 114 is operated to collect the cell suspension in the culture vessel 103. After that, the culture medium is injected from the liquid feeding mechanism 108 to entrain the detached cells remaining in the culture vessel 103, and the cell collection mechanism 114 is used to collect the remaining cells. In a case of cells having a strong adhesion force, it is also effective to introduce a flow in which the image pickup apparatus 107 is used to examine the state of the depression 102 subjected to the detachment and, when a detachment residue is identified, the corresponding depression 102 is again subjected to the spraying of the PBS and the collection of the cells.

As described above, in the third embodiment, there has been described a cell culture system including: a culture substrate in which a plurality of depressions are arranged and to which cells are caused to adhere; a scraper configured to detach cells adhering to a region other than the plurality of depressions on the culture substrate by sliding on the culture substrate against the region other than the plurality of depressions; and a cell removal mechanism configured to remove, from the culture substrate, the cells detached by the scraper.

Accordingly, it is possible to provide a cell culture system capable of automatically culturing and collecting a desired amount of cells. Further, in the third embodiment, there is provided a cell culture method including: culturing cells by causing the cells to adhere to a culture substrate in which a plurality of depressions are arranged; detaching cells adhering to a region other than the plurality of depressions on the culture substrate by causing a scraper to slide on the culture substrate against the region other than the plurality of depressions; and removing, from the culture substrate, the cells detached by the scraper.

The third embodiment has been verified through use of iPS cells, but most types of cell can be used as long as the cells are adherent cells. In that case, types of culture medium and reagent and a timing of the detachment may be changed as appropriate in accordance with characteristics of each cell and a protocol to be implemented.

In the present invention, cells that are cultured on a culture substrate can be referred to as cells to be cultured, cells that adhere to a region other than depressions can be referred to as cells to be removed, and cells that adhere to the inside of a depression can be referred to as cells to be collected.

According to the present invention, it is possible to provide the cell culture system that facilitates the estimation of the number of cells present on the culture substrate after the cell culture.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2023-091759, filed Jun. 2, 2023, which is hereby incorporated by reference herein in its entirety.

What is claimed is:

1. A cell culture system comprising:
a culture substrate in which a plurality of depressions are arranged and to which cells are caused to adhere;
a scraper configured to detach cells adhering to a region other than the plurality of depressions on the culture substrate by sliding on the culture substrate against the region other than the plurality of depressions; and
a cell removal mechanism configured to remove, from the culture substrate, the cells detached by the scraper.

2. A cell culture system comprising:
a culture substrate in which a plurality of depressions are arranged and to which extracellular matrices are caused to adhere;
a scraper configured to detach extracellular matrices adhering to a region other than the plurality of depressions on the culture substrate by sliding on the culture substrate against the region other than the plurality of depressions; and
an extracellular matrix removal mechanism configured to remove, from the culture substrate, the extracellular matrices detached by the scraper,
wherein the culture substrate is configured to cause cells to adhere to the culture substrate through intermediation of the extracellular matrices.

3. The cell culture system according to claim 1, further comprising an image pickup apparatus configured to allow observation of a state of cells.

4. The cell culture system according to claim 3, further comprising an information processing unit including:
a cell determination function of determining a state of cells in each of the plurality of depressions based on the state of cells acquired by the image pickup apparatus; and
a depression selection function of selecting one of the plurality of depressions to which the cells to be detached adhere based on the state of cells in each of the plurality of depressions which has been determined by the cell determination function.

5. The cell culture system according to claim 1, further comprising an image pickup apparatus configured to allow observation of a detachment residue of cells adhering to the region other than the plurality of depressions.

6. The cell culture system according to claim 5, further comprising an information processing unit configured to operate, when the detachment residue is identified, the scraper so as to remove the detachment residue.

7. The cell culture system according to claim 1, wherein a width of the scraper in a direction perpendicular to a sliding axis of the scraper on the culture substrate is larger than a width of each of the plurality of depressions in the direction perpendicular to the sliding axis of the scraper on the culture substrate.

8. The cell culture system according to claim 1, wherein the plurality of depressions each has an inclined surface.

9. The cell culture system according to claim 1, wherein the plurality of depressions each has a recessed curved surface.

10. The cell culture system according to claim 1, wherein the region other than the plurality of depressions has a protruding curved surface.

11. The cell culture system according to claim 1, further comprising:
a cell detachment mechanism configured to detach cells from the plurality of depressions; and
a cell collection mechanism configured to collect the cells detached by the cell detachment mechanism.

12. The cell culture system according to claim 1, further comprising a cell culture mechanism configured to culture cells.

13. The cell culture system according to claim 1, wherein the cells comprise adherent cells.

14. A cell culture method comprising:
culturing cells by causing the cells to adhere to a culture substrate in which a plurality of depressions are arranged;
detaching cells adhering to a region other than the plurality of depressions on the culture substrate by causing a scraper to slide on the culture substrate against the region other than the plurality of depressions; and
removing, from the culture substrate, the cells detached by the scraper.

15. A cell culture method comprising:
applying extracellular matrices to a culture substrate in which a plurality of depressions are arranged;
detaching, after the extracellular matrices have been applied, extracellular matrices adhering to a region other than the plurality of depressions on the culture substrate by causing a scraper to slide on the culture substrate against the region other than the plurality of depressions;
removing, from the culture substrate, the extracellular matrices detached by the scraper; and
seeding cells on the culture substrate subjected to the removing, and culturing the cells.
